(19) United States
(12) Patent Application Publication
(10) Pub. No.: US 2025/0322959 A1
(43) Pub. Date: Oct. 16, 2025

(54) DECISION SUPPORT TOOL FOR MYOCARDIAL INFARCTION, SYSTEM AND METHOD

(71) Applicant: The University Court of the University of Edinburg, Edinburg (GB)

(72) Inventors: Dimitrios DOUDESIS, Edinburgh (GB); Kuan Ken LEE, Edinburgh (GB); Nicholas Linton MILLS, Edinburgh (GB)

(73) Assignee: The University Court of the University of Edinburg, Edinburg (GB)

(21) Appl. No.: 18/873,296

(22) PCT Filed: Aug. 25, 2023
(86) PCT No.: PCT/GB2023/052209

§ 371 Date: Dec. 9, 2024

(30) Foreign Application Priority Data
Aug. 26, 2022 (GB) 2212464.8

(51) Int. Cl. G16H 50/30; A61B 5/00; G16B 40/20

(52) U.S. Cl. CPC G16H 50/30; G16B 40/20; A61B 5/7275

(57) ABSTRACT

A computer implemented method of providing and an indication of the probability of myocardial infarction using cardiac biomarker measurements comprises combining measured with other clinical indicators in statistical model to compute the probability of a subject having suffered myocardial infarction, the statistical model using a machine learning algorithm. A decision tool and a system for implementing the method are disclosed.

### FIELD OF THE DISCLOSURE

The present disclosure relates to a decision support tool, system and method. Particularly, but not exclusively, it relates to decision support tool, system and method to provide an indication of the probability of myocardial infarction in a subject. More particularly, but not exclusively, it relates to a tool, system and method to provide an indication of the probability of myocardial infarction using one or more cardiac biomarker measurements.

### BACKGROUND TO THE DISCLOSURE

Myocardial infarction is a condition characterised by myocardial necrosis secondary to acute myocardial ischaemia and is the most common cause of death worldwide. Consequently, the rapid diagnosis of myocardial infarction is critically important to improve outcome for subjects who may, or may not, have suffered from the condition.

In view of this diagnostic pathways have been developed to evaluate the release of biomarkers into the bloodstream by tissue damaged by myocardial necrosis. Exemplary assay biomarkers used for this purpose include, but are not limited to, creatine-kinase-MB isoform, cardiac myosin binding protein C and cardiac troponin with troponin being the preferred biomarker.

The use of high-sensitivity cardiac troponin assays have led to the development and widespread adoption of accelerated diagnostic pathways to expedite the assessment of subjects with suspected acute coronary syndrome but have important limitations.

Firstly, they use fixed cardiac troponin thresholds for all subjects, which do not account for age, sex or comorbidities which are known to influence troponin concentrations, for example kidney disease can lead to an increase in troponin levels.

Secondly, they are based on set time points for serial testing and rely on fixed changes in troponin levels at specific time intervals to indicate whether a subject had had a myocardial infarction or not. The delay between troponin measurements can be of the order of hours thereby delaying the diagnosis of myocardial infarction and potentially leading to a worse outcome for the subject. Additionally, there are challenges in adhering to specific time bounded measurements in a busy Emergency Department, and consequently, such pathways may not be generalisable to all health care systems.

Thirdly, they broadly categorise subjects as either low-, intermediate-or high-risk based on troponin thresholds alone, and do not consider other important information, such as the time of symptom onset or findings on the electrocardiogram. Troponin levels can be elevated from pre-existing injury to the heart or as noted hereinbefore by kidney disease, an infection or other comorbidity.

An existing algorithm, the myocardial-ischemic-injury-index (MI³), was developed using gradient boosting, to compute an individualised probability of myocardial infarction for subjects with suspected acute coronary syndrome. Whilst this algorithm overcomes several issues with fixed cardiac troponin thresholds, there are important limitations that may limit its implementation. Firstly, MI³ requires serial cardiac troponin measurements for both the rule-in and rule-out of myocardial infarction which precludes the use of this algorithm during the initial subject assessment. This would significantly limit the efficiency of MI³ since assessment pathways for subjects with suspected acute coronary syndrome currently recommend the use of a single cardiac troponin measurement at presentation to risk stratify subjects; an approach that has been shown to be safe and effective at shortening the duration of stay. Secondly, the MI³ score is calculated using only age, sex and cardiac troponin concentration. Although the use of these limited and widely available variables may facilitate its implementation due to simplicity, this has also limited its diagnostic performance by not including other important subject factors that influence cardiac troponin. Moreover, specificity and positive predicted value was significantly lower in important subgroups such as older subjects, women and those with significant comorbidities such as chronic kidney disease. Finally, MI³ was developed in a relatively small cohort of selected subjects. A recently performed an external validation of the MI³ algorithm and observed it had poor calibration when applied to a cohort of consecutive subject with suspected acute coronary syndrome. CoDE-ACS overcomes these limitations by including other subject factors that influence cardiac troponin concentration, allowing the use of a single measure of cardiac troponin at presentation and by training the model in a large unselected subject population. Removing the need for multiple troponin measurements at specific timepoints and the inclusion of multiple clinical variables improves both the speed and accuracy of diagnosis of myocardial infarction.

### SUMMARY OF THE DISCLOSURE

According to a first aspect of the present disclosure there is provided a computer implemented method of identifying an subject's likelihood of having myocardial infarction comprising the steps of operating upon data corresponding to the level of a cardiac biomarker in at least one sample from an individual with at least two other data elements indicative of respective clinical indicators from the individual in a statistical model to compute the probability of myocardial infarction for the individual wherein the data corresponding to the level of the cardiac biomarker is provided as a discrete variable in the model.

Such a method provides for the use of a single troponin measurement to identify a subject's likelihood of having myocardial infarction. This removes the requirement for multiple troponin measurements over an extended time period, for example as in the existing MI³ algorithm, thereby simplifying the procedure and reducing the time for a diagnosis and leading to an improved outcome for a subject. The present disclosure overcomes the limitations of the prior art by including other subject factors that influence cardiac troponin concentration, allowing the use of a single measure of cardiac troponin at presentation and by training the model in a large unselected subject population. The present disclosure can appropriately risk-stratifying subjects at presentation who are likely to benefit from further specialist investigation and treatment.

The cardiac biomarker may comprise cardiac troponin I and/or cardiac troponin T, natriuretic peptides and/or cardiac myosin binding protein C measured using point of care and/or core lab assays.

The data corresponding to the level of the cardiac biomarker in at least one sample from an individual may comprise data corresponding to the level of the cardiac biomarker in a single sample.

The clinical parameters may comprise at least two data elements are selected from the list comprising: age, sex, the number of hours from symptom onset to cardiac biomarker measurement, presenting symptoms, prior medical diagnoses, such as known ischaemic heart disease, hyperlipidemia and other risk factors, heart rate, blood pressure, Killip class, information from an electrocardiogram, renal function, haemoglobin and other information from laboratory testing or imaging. Renal function may be estimated by glomerular filtration rate calculated using the Chronic Kidney Disease Epidemiology Collaboration formula.

The method may comprise loading respective training data sets corresponding to subjects with and without myocardial injury into a machine learning system wherein the machine-learning system includes a processing circuitry arranged to be trained to myocardial infarction using the training data within the statistical model.

The method may comprise using the machine learning system to execute the statistical model on the data corresponding to the level of the cardiac biomarker in at least one sample from an individual with at least two other data elements indicative of respective clinical indicators from the individual once trained.

The statistical model may comprise an XGBoost model from the boosting family of models or a random forest model from the bagging family of models or artificial and/or convolutional neural networks models or logistic regression or generalised linear mixed models wherein a probability that is computed by performing an inverse-logit transformation of the sum of the weights of the terminal nodes of the trained model, the XGBoost model

$${\hat{y}}_{i} = {\sum }_{k = 1}^{K} {f}_{k} ( {x}_{i} ) , {f}_{k} ∈ F$$

where f is an function that map each variable vector xᵢ (xᵢ={xᵢ, x₂, . . . , xₙ}, i=1, 2, N) to the outcome yᵢ, K is the number of Classification and Regression Trees (CART) and F is the space of function containing all CART

The XGBoost may optimise an objective function of the form:

$$Obj = {\sum }_{i = 1}^{N} l ( {y}_{i} , {\hat{y}}_{i} ) + {\sum }_{k = 1}^{K} Ω ( {f}_{k} )$$

Where the first term is a loss function, l, which evaluates how well the model fits the data by measuring the difference between the prediction ŷᵢ and the outcome yᵢ.

The method may comprise training the machine learning system by performing a plurality of iterations of 10-fold cross-validation respective training data sets corresponding to subjects with and without myocardial injury to compute a score to indicative of the probability of having myocardial infarction for each individual in the respective training data sets.

The method may comprise generating a probability score for an individual that would classify the individual as a high-, intermediate- or a low-probability of myocardial infarction. The method may comprise defining one or more user variable predictor values based upon a user input. The one or more user variable predictor variables may define thresholds for classifying an individual as a high- or a low-probability of myocardial infarction.

According to a second aspect of the present disclosure there is provided a system for identifying a subject's likelihood of having myocardial infarction comprising:
a processor;
a data storage device; and
an output device;
wherein the processor is arranged to receive data corresponding to the level of a cardiac biomarker in at least one sample from an individual with at least two other data elements indicative of respective clinical indicators from the individual in a statistical model from the data storage device and to execute a set of instructions to cause the processor to:
   operate upon data corresponding to the level of the cardiac biomarker in the at least one sample from an individual with the at least two other data elements indicative of respective clinical indicators from the individual in a statistical model to compute the probability of myocardial infarction for the individual wherein the data corresponding to the level of the cardiac biomarker is provided as a discrete variable in the model; and
   output data corresponding to the likelihood of the individual having myocardial infarction at the output device.

The cardiac biomarker may comprise cardiac troponin I and/or cardiac troponin T, natriuretic peptides and/or cardiac myosin binding protein C measured using point of care and/or core laboratory assays.

The data corresponding to the level of the cardiac biomarker in at least one sample from an individual may comprise data corresponding to the level of the cardiac biomarker in a single sample.

The clinical parameters may comprise at least two data elements are selected from the list comprising: age, sex, the number of hours from symptom onset to cardiac biomarker measurement, presenting symptoms, prior medical diagnoses, such as known ischaemic heart disease, hyperlipidemia and other risk factors, heart rate, blood pressure, Killip class, information from an electrocardiogram, renal function, haemoglobin and other information from laboratory testing or imaging.

The processor may be arranged to load respective training data sets corresponding to subjects with and without myocardial injury from the data storage device into a machine learning sub-system system wherein the machine-learning sub-system system includes a processing circuitry arranged to be trained to myocardial infarction using the training data within the statistical model.

The machine learning system to may be arranged to execute instructions that cause the statistical model to be executed on the data corresponding to the level of troponin in at least one sample from an individual with at least two other data elements indicative of respective clinical indicators from the individual once the machine learning sub-system is trained.

The statistical model may comprise an XGBoost model wherein a probability that is computed by performing an inverse-logit transformation of the sum of the weights of the terminal nodes of the trained model, the XGBoost model

$${\hat{y}}_{i} = {\sum }_{k = 1}^{K} {f}_{k} ( {x}_{i} ) , {f}_{k} ∈ F$$

where f is an function that map each variable vector xᵢ (xᵢ={xᵢ, x₂, . . . , xₙ}, l=1, 2, N) to the outcome yᵢ, K is the number of Classification and Regression Trees (CART) and F is the space of function containing all CART

The XGBoost may optimise an objective function of the form:

$$Obj = {\sum }_{i = 1}^{N} l ( {y}_{i} , {\hat{y}}_{i} ) + {\sum }_{k = 1}^{K} Ω ( {f}_{k} )$$

Where the first term is a loss function, i, which evaluates how well the model fits the data by measuring the difference between the prediction ŷᵢ and the outcome yᵢ.

The machine learning system sub-system may be arranged to be trained by performing a plurality of iterations of 10-fold cross-validation respective training data sets corresponding to subjects with and without myocardial injury to compute a score to indicative of the probability of having myocardial infarction for each individual in the respective training data sets.

The processor may be arranged to generate a probability score for an individual that would classify the individual as a high-, intermediate- or a low-probability of myocardial infarction. The processor may be arranged to define one or more user variable predictor values based upon a user input. The one or more user variable predictor variables may define thresholds for classifying an individual as a high-or a low-probability of myocardial infarction.

According to a third aspect of the present disclosure there is provided a processor arranged to execute the method of the first aspect of the present disclosure or to act as the processor of the second aspect of the present disclosure.

According to a fourth aspect of the present disclosure there is provided a computer implemented tool capable of receiving data to allow establishment or the ruling out of a risk of myocardial infarction comprising the processor of the third aspect of the present disclosure.

Such a decision support tool provides excellent discrimination in the internal and external validation cohorts and exhibits consistent performance across subject subgroups compared to fixed guideline-recommended thresholds of cardiac troponin compared to previous diagnostic algorithms.

### BRIEF DESCRIPTION OF THE DRAWINGS

The present disclosure will now be described, by way of example only, with reference to the accompanying drawings in which:

FIG. 1 is a schematic representation of a system for identifying a subject's likelihood of having myocardial infarction according to an aspect of the present disclosure;

FIG. 2 is a flow diagram of the analysis of a subject population;

FIG. 3 is a graphical representation of negative predictive value of the 2 ng/L cardiac troponin threshold in the derivation cohort across patient subgroups;

FIG. 4 is a graphical representation of negative predictive value of the 5 ng/L cardiac troponin threshold in the derivation cohort across patient subgroups;

FIG. 5 is a graphical representation of positive predictive value of the sex-specific 99^{th} centile cardiac troponin threshold in the derivation cohort across patient subgroups;

FIG. 6 is a graphical representation of positive predictive value of the 64 ng/L cardiac troponin threshold in the derivation cohort across patient subgroups;

FIG. 7a is a graphical representation of diagnostic performance of the CoDE-ACS score using presentation cardiac troponin concentration in the derivation cohort across patient subgroups—CoDE-ACS rule-out score;

FIG. 7b is a graphical representation of diagnostic performance of the CoDE-ACS score using presentation cardiac troponin concentration in the derivation cohort across patient subgroups—CoDE-ACS rule-in score;

FIG. 8a is a graphical representation of diagnostic performance of the CoDE-ACS score using serial cardiac troponin concentrations across patient subgroups in the derivation cohort—CoDE-ACS rule-out score;

FIG. 8b is a graphical representation of diagnostic performance of the CoDE-ACS score using serial cardiac troponin concentrations across patient subgroups in the derivation cohort—CoDE-ACS rule-in score;

FIGS. 9a is graphical representations of diagnostic performance of the CoDE-ACS score in the external validation cohort using the presentation troponin concentration alone—Receiver-operating-characteristic (ROC) curve illustrating discrimination of the CODE-ACS for myocardial infarction;

FIGS. 9b is graphical representations of diagnostic performance of the CoDE-ACS score in the external validation cohort using the presentation troponin concentration alone—calibration of the CoDE-ACS score with the observed proportion of patients with myocardial infarction (The dashed line represents perfect calibration and each point represents 100 patients);

FIG. 10a is a graphical representation of diagnostic performance of the CoDE-ACS in the external validation cohort score using serial troponin results—receiver-operating-characteristic (ROC) curve illustrating discrimination of the CoDE-ACS for myocardial infarction;

FIG. 10b is a graphical representation of diagnostic performance of the CODE-ACS in the external validation cohort score using serial troponin results—Calibration of the CODE-ACS score with the observed proportion of patients with myocardial infarction (The dashed line represents perfect calibration and each point represents 100 patients);

FIG. 11a is a graphical representation of diagnostic performance of the CoDE-ACS score across patient subgroups in the external validation cohort—negative predictive value of CODE-ACS using the presentation troponin concentration alone across patient subgroups;

FIG. 11b is a graphical representation of diagnostic performance of the CoDE-ACS score across patient subgroups in the external validation cohort—positive predictive value of CODE-ACS using the presentation troponin concentration alone across patient subgroups;

FIG. 12a is a graphical representation of diagnostic performance of the CoDE-ACS score in the external validation cohort using serial cardiac troponin concentrations across patient subgroups in the external validation—CoDE-ACS rule-out score;

FIG. 12a is a graphical representation of diagnostic performance of the CoDE-ACS score in the external validation cohort using serial cardiac troponin concentrations across patient subgroups in the external validation—CoDE-ACS rule-in score;

FIG. 13 is a flow chart showing performance of CODE-ACS as a pathway in the external validation cohort;

FIG. 14a is a graph showing cumulative incidence of mortality in the external validation cohort stratified by CoDE-ACS probability group—all-cause mortality;

FIG. 14b is a graph showing cumulative incidence of mortality in the external validation cohort stratified by CoDE-ACS probability group—cardiac mortality;

FIG. 15a is a cumulative incidence plot stratified by CoDE-ACS probability groups after serial measurements in the external validation cohort—all-cause mortality; and

FIG. 15b is a cumulative incidence plot stratified by CoDE-ACS probability groups after serial measurements in the external validation cohort—cardiac mortality.

### DETAILED DESCRIPTION

The present machine learning algorithm for the diagnosis of myocardial infarction allows the use of cardiac troponin or other cardiac biomarker concentrations at presentation or on serial testing and incorporates important subject factors that influence cardiac biomarkers. It exhibits excellent discrimination and good calibration in the derivation and external validation cohorts with consistent performance across subgroups. Subjects identified as low-probability of myocardial infarction on the index visit had a very low-probability of dying from cardiac diseases or other causes at one year.

An evaluation of the diagnostic performance of guideline-recommended cardiac troponin thresholds across important subject subgroups is described as well as a decision-support tool that uses machine learning to calculate the probability of myocardial infarction for each subject. An external validation of the performance of the accompanying decision support tool is described.

The High-Sensitivity Troponin in the Evaluation of Subjects With Suspected Acute Coronary Syndrome (High-STEACS; NCT01852123) trial population is used as the derivation cohort to develop machine learning model. High-STEACS is a stepped-wedged cluster-randomised controlled trial that evaluated the implementation of a high-sensitivity cardiac troponin I assay in consecutive subjects with suspected acute coronary syndrome presenting to 10 secondary and tertiary hospitals in Scotland between Jun. 10, 2013, and Mar. 3, 2016. The trial design has been described previously, see Shah ASV, Anand A, Strachan FE, et al. High-sensitivity troponin in the evaluation of subjects with suspected acute coronary syndrome: a stepped-wedge, cluster-randomised controlled trial. The Lancet 2018; 392(10151): 919-28, the contents of which are hereby incorporated by reference.

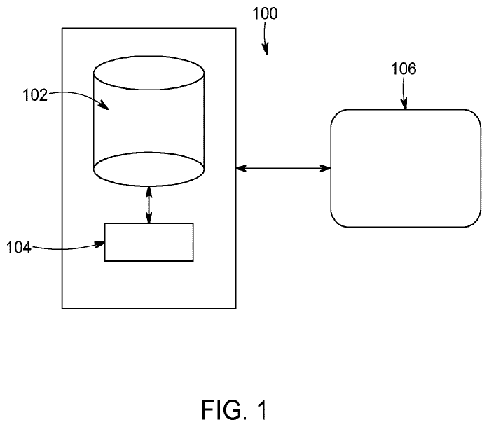

Referring now to FIG. 1, a system (100) for identifying a subject's likelihood of having myocardial infarction comprises a processor (102), a data storage device (104) and an display unit (106).

The data storage device (104) has data corresponding to single troponin or other cardiac biomarker measurements a derivation cohort consisting of a plurality of subjects with an adjudicated diagnosis of myocardial infarction and without evidence of myocardial injury. In addition to this troponin measurement the data storage device (104) stores data corresponding to a plurality of clinical indicators for each subject. Non-limiting examples of clinical indicators are: age, sex, the number of hours from symptom onset to cardiac biomarker measurement, presenting symptoms, prior medical diagnoses, such as known ischaemic heart disease, hyperlipidemia and other risk factors, heart rate, blood pressure, Killip class, information from an electrocardiogram, renal function, haemoglobin and other information from laboratory testing or imaging. Renal function may be estimated by glomerular filtration rate calculated using the Chronic Kidney Disease Epidemiology Collaboration formula. The troponin or other cardiac biomarker measurement data and the other clinical indicator data comprise training data for a machine learning algorithm for the diagnosis of myocardial infarction, (hereinafter referred to as “CoDE-ACS”), that is loaded on to the processor (102). It will be appreciated that the same clinical indicators need not be loaded into the CoDE-ACS for each subject and thus the CoDE-ACS is robust and can accommodate variable data inputs and does not require the same clinical indicators across all subjects.

When executed, the CoDE-ACS employs a statistical model may comprising an XGBoost model wherein a probability that is computed by performing an inverse-logit transformation of the sum of the weights of the terminal nodes of the trained model, the XGBoost is described in detail hereinafter.

In the present embodiment, the CoDE-ACS is trained by performing, by way of non-limiting example, ten of iterations of 10-fold cross-validation respective training data sets corresponding to subjects with and without myocardial injury to compute a score to indicative of the probability of having myocardial infarction for each individual in the respective training data sets. It will be appreciated that other numbers of iterations can be executed.

When in use for decision support, the processor (102) is loaded with data corresponding a single troponin or other cardiac biomarker measurement of a test subject, along with data corresponding to at least two clinical indicators of the type, by way of non-limiting example, listed hereinbefore.

The trained CoDE-ACS is executed on the test subject data and generates a probability score for an individual that would classify the individual as a high-, intermediate- or a low-probability of myocardial infarction. This probability can be used as an indicator of whether further investigation of the test subject's condition is required or not and also whether they fall in an intermediate classification where the risk may be managed in an alternative manner. Typically, the probability and rule-in/rule-out thresholds are displayed graphically upon the display unit (106).The rule-in/rule-out thresholds can be user defined using either a graphical user interface or a text based interface displayed on the display unit (106).

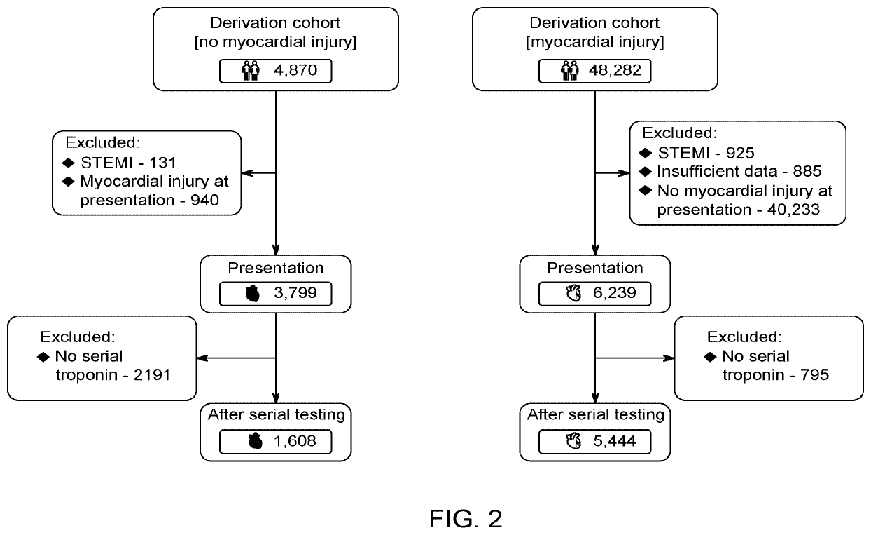

Subjects were included in a prespecified secondary analysis based on the following criteria: (1) age ≥18 years old, (2) presentation with suspected acute coronary syndrome, (3) high-sensitivity cardiac troponin measurement, (4) availability of electrocardiogramd physiological data for diagnostic adjudication. Subjects a diagnosis of ST-segment elevation myocardial infarction were excluded given they undergo coronary revascularisation directly without troponin testing in the Emergency Department, see for example FIG. 2.

A machine learning model is used to predict an adjudicated diagnosis of myocardial infarction during the index hospital admission.

### Performance of Guideline Recommended Cardiac Troponin Thresholds

The diagnostic performance and proportion of subjects identified by guideline recommended cardiac troponin thresholds to rule-out and rule-in myocardial infarction were evaluated. These were evaluated in the overall population and in key pre-specified subgroups by age, sex, time from symptom onset to troponin measurement, renal impairment, prior ischaemic heart disease, diabetes mellitus, and cerebrovascular disease.

### Feature Selection and Processing

High-sensitivity cardiac troponin concentrations were used as a continuous measure and clinical variables known to be associated with cardiac troponin or myocardial infarction, which were found to have the highest relative importance in our model training phase. These were age, sex, the number of hours from symptom onset to cardiac biomarker measurement, presenting symptoms, prior medical diagnoses, such as known ischaemic heart disease, hyperlipidemia and other risk factors, heart rate, blood pressure, Killip class, information from an electrocardiogram, renal function, haemoglobin and other information from laboratory testing or imaging. To maximise the clinical utility of our models, models using cardiac troponin or another cardiac biomarker concentration at presentation alone are used. Subsequently developed models can be used to include a second or third cardiac troponin or another cardiac biomarker concentration, measured at an early and flexible timepoint.

### Model Development and Validation

Many statistical models were evaluated in the development of the decision-support tool-logistic regression, naïve bayes, random forest and extreme gradient boosting (XGBoost). The XGBoost algorithm was selected and developed using the R package ‘xgboost’

XGBoost is a supervised machine learning technique initially proposed by Chen and Guestrin. In brief, gradient boosting employs an ensemble technique to iteratively improve model accuracy for regression and classification problems. This ensemble-based algorithm is achieved by creating sequential models, using decision trees as learners where subsequent models attempt to correct errors of the preceding models. In the boosting method, individuals that were misclassified by the previous model are assigned a higher weight to increase their chance of being selected in subsequent models. Each model is subsequently fitted in a step-wise fashion to minimise loss function such as absolute error or squared error (the amount predicted values differ from the true values). XGBoost refers to the re-engineering of gradient boosting to significantly improve the speed of the algorithm by pushing the limits of computational resources. The output of the XGBoost model is a probability that is computed by performing an inverse-logit transformation of the sum of the weights of the terminal nodes of the trained model.

The mathematical formula for the gradient boosting model can be described as:

$${\hat{y}}_{i} = {\sum }_{k = 1}^{K} {f}_{k} ( {x}_{i} ) , {f}_{k} ∈ F \qquad ( 1 )$$

where f is an function that map each variable vector xᵢ (xᵢ={xᵢ, x₂, . . . , xₙ}, i=1, 2, N) to the outcome yᵢ, K is the number of Classification and Regression Trees (CART) and F is the space of function containing all CART.

XGBoost optimises an objective function of the form:

$$Obj = {\sum }_{i = 1}^{N} l ( {y}_{i} , {\hat{y}}_{i} ) + {\sum }_{k = 1}^{K} Ω ( {f}_{k} ) \qquad ( 2 )$$

Where the first term is a loss function, l, which evaluates how well the model fits the data by measuring the difference between the prediction ŷᵢ and the outcome yᵢ. The second term, the regularization term, is used by XGBoost to avoid overfitting by penalizing the complexity of the model. Furthermore, to improve and fully leverage the advantages of XGBoost the hyper-parameters of the algorithm defined below were tuned through a grid search strategy using 10-fold cross-validation.

### Table 1. The Hyper-Parameter Values Used to Develop the CoDE-ACS

Separate models are used for those with and without myocardial injury (for example a cardiac troponin I concentration above the sex-specific 99^{th} centile threshold; 16 ng/L in women and 34 ng/L in men). For both models, in the derivation cohort imputed ten datasets are multiplied to account for missing data and performed ten iterations of 10-fold cross-validation to compute a score (0-100) that corresponds to an individual subject's probability of having myocardial infarction. The scores that would classify the highest proportion of subjects as high- or low-probability are identified using prespecified criteria to optimise performance to rule-in (80% PPV and 80% specificity) myocardial infarction in those with myocardial injury and to rule-out (99.5% NPV and 95% sensitivity) myocardial infarction in those without myocardial injury. The model with the best diagnostic performance in the overall population and in pre-specified subject subgroups was selected for the decision-support tool.

The decision support tool has been validated using the IMPACT (Improved Assessment of Chest pain Trial), ADAPT (2-Hour Accelerated Diagnostic Protocol to Assess Subjects With Chest Pain Symptoms Using Contemporary Troponins as the Only Biomarker) and SPACE (Signal Peptide in Acute Coronary Events) cohorts from Australia and New Zealand. All analyses were performed in R version 4.0.3.

| | Presentation | | Using serial | |
|---|---|---|---|---|
| | troponin | | troponin | |
| | concentration alone | | concentration | |
| | Rule-out | Rule-in | Rule-out | Rule-in |
| number of iterations | 83 | 199 | 72 | 184 |
| learning rate | 0.11 | 0.14 | 0.35 | 0.05 |
| interaction depth | 3 | 2 | 6 | 6 |
| minimum number of observations | 5 | 2 | 6 | 7 |
| in the terminal nodes | | | | |
| fraction of the training set | 0.56 | 0.62 | 0.90 | 0.65 |
| observations randomly selected | | | | |
| for each subsequent tree | | | | |
| fraction of variables randomly | 0.83 | 0.88 | 0.89 | 0.90 |
| sampled for each tree | | | | |

The derivation cohort consists of 10,038 subjects (median age 70 years, 48% women). In this cohort, during the index hospitalisation, the adjudicated diagnosis was myocardial infarction in 49% (3,062/6,239) and 3% (132/3,799) in subjects with and without evidence of myocardial injury at presentation.

### Table 2. Baseline Characteristics of the Derivation and External Validation Cohorts

| | Derivation cohort | | External validation cohort | |
|---|---|---|---|---|
| | No | | No | |
| | myocardial | Myocardial | myocardial | Myocardial |
| | injury at | injury at | injury at | injury at |
| | presentation | presentation | presentation | presentation |
| Number of subjects | 3,799 | 6,239 | 2,656 | 379 |
| Age, years | 62 (50, 74) | 74 (62, 83) | 55 (46, 66) | 70 (59,79) |
| Sex | | | | |
| Female | 1,580 (42%) | 3,199 (51%) | 1,039 (39%) | 154 (41%) |
| Male | 2,219 (58%) | 3,040 (49%) | 1,617 (61%) | 225 (59%) |
| Chest pain at presentation | 3,251 (86%) | 4,030 (70%) | 2,550 (96%) | 366 (97%) |
| Early presenter (≤2 hours | 606 (16%) | 1,209 (21%) | 588 (22%) | 37 (10%) |
| from symptom onset) | | | | |
| Previous medical conditions | | | | |
| Myocardial infarction | 606 (18%) | 837 (13%) | 493 (19%) | 127 (24%) |
| Ischemic heart disease | 1,133 (34%) | 2,136 (34%) | 618 (23%) | 158 (42%) |
| Cerebrovascular disease | 236 (7%) | 626 (10%) | 142 (5%) | 36 (9%) |
| Diabetes mellitus | 513 (16%) | 919 (15%) | 340 (13%) | 86 (23%) |
| Previous revascularisation | | | | |
| PCl | 360 (11%) | 560 (9%) | 399 (15%) | 88 (23%) |
| CABG | 178 (5%) | 161 (3%) | 164 (6%) | 45 (12%) |
| Medications at presentation | | | | |
| Aspirin | 720 (30%) | 2,267 (36%) | 861 (32%) | 187 (49%) |
| Dual anti-platelet therapy† | 115 (5%) | 309 (5%) | 22 (1%) | 3 (1%) |
| ACE or ARB | 745 (31%) | 2,681 (43%) | 335 (13%) | 37 (10%) |
| Beta-blocker | 584 (25%) | 2,156 (35%) | 236 (9%) | 36 (10%) |
| Electrocardiogram result§ | | | | |
| Normal | 3,279 (87%) | 3,308 (64%) | 1,197 (45%) | 33 (10%) |
| Myocardial ischaemia | 458 (14%) | 1,351 (26%) | 137 (5%) | 110 (29%) |
| ST segment elevation | 93 (3%) | 196 (4%) | 18 (1%) | 10 (3%) |
| Physiological parameters§ | | | | |
| Heart rate, beats per minute | 77 (65, 90) | 81 (68,99) | 73 (64, 84) | 75 (65, 88) |
| Systolic blood pressure, mmHg | 137 (121, 153) | 138 (120, 157) | 138 (124, 155) | 144 (128, 162) |
| Haematology and clinical | | | | |
| chemistry measurements | | | | |
| Haemoglobin, g/L | NA | 133 (118, 146) | 143 (133, 153) | 138 (124, 151) |
| eGFR, ml/min | 86 (69, 99) | 66 (44, 85) | 83 (69, 90) | 67 (49, 83) |
| Presentation high-sensitivity | 3 (2, 7) | 85 (41, 320) | 3 (2, 5) | 130 (46, 660) |
| cardiac troponin I | | | | |
| Serial high-sensitivity | 5 (3, 11) | 209 (53, 1,786) | 3 (2, 5) | 192 (56, 1,114) |
| cardiac troponin I, % ¶ | | | | |
| Peak high-sensitivity | 4 (2, 8) | 170 (51, 1,422) | 3 (2, 5) | 198 (58, 1,127) |
| cardiac troponin I, ng/L | | | | |
| Adjudicated diagnosis | | | | |
| Type 1, 4b or 4c MI | 132 (3%) | 3,062 (49%) | 63 (2%) | 271 (72%) |
| Type 2 MI | 33 (1%) | 802 (13%) | 39 (1%) | 34 (9%) |
| Non-ischemic myocardial injury | 21 (1%) | 2,343 (38%) | 24 (1%) | 74 (20%) |

| | True | False | True | False | NPV | Sensitivity | Proportion |
|---|---|---|---|---|---|---|---|
| | negative | negative | positive | positive | (95% CI) | (95% CI) | ruled out |
| Cardiac troponin | | | | | | | |
| thresholds | | | | | | | |
| <2 ng/L | 606 | 3 | 129 | 3061 | 99.5 | 97.7 | 16.0% |
| | | | | | (98.9-100.0) | (94.7-100.0) | |
| <5 ng/L | 2286 | 9 | 123 | 1381 | 99.6 | 93.2 | 60.4% |
| | | | | | (99.3-99.8) | (88.8-97.2) | |
| CoDE-ACS score | | | | | | | |
| Presentation | 2510 | 5 | 127 | 1157 | 99.8 | 96.2 | 66.2% |
| (1.2) | | | | | (99.6-100.0) | (92.3-99.2) | |
| Serial testing | 1118 | 2 | 130 | 341 | 99.8 | 98.5 | 70.4% |
| (0.6) | | | | | (99.5-100.0) | (96.1-100.0) | |

### 8. Table 3. Diagnostic Performance of Guideline-Recommended Rule-In and Rule-Out Thresholdis for Myocardial Infarction and CoDE-ACS in the Derivation Cohort Rule-Out Thresholds in Subjects Without Myocardial Injury at Presentation

B. Rule-in thresholds in subjects with myocardial injury at presentation

| | True | False | True | False | PPV | Specificity | Proportion |
|---|---|---|---|---|---|---|---|
| | negative | negative | positive | positive | (95% CI) | (95% CI) | ruled in |
| Cardiac troponin | | | | | | | |
| thresholds | | | | | | | |
| >99^{th} centile | 0 | 0 | 3085 | 3154 | 49.4 | 0 | 100.0% |
| | | | | | (48.2-50.7) | (0-0) | |
| ≥64 ng/L | 1657 | 952 | 2133 | 1497 | 58.8 | 52.5 | 58.2% |
| | | | | | (57.2-60.3) | (50.8-54.1) | |
| CoDE-ACS score | | | | | | | |
| Presentation | 2620 | 945 | 2140 | 534 | 80.0 | 83.1 | 42.9% |
| (59.9) | | | | | (78.4-81.5) | (81.7-84.4) | |
| Serial testing | 1851 | 854 | 2278 | 461 | 83.2 | 80.1 | 50.3% |
| (62.9) | | | | | (81.7-84.4) | (78.4-81.6) | |

### Diagnostic Performance of Cardiac Troponin Thresholds in Subgroups

In the derivation cohort, in those without myocardial injury at presentation the negative predictive value of cardiac troponin rule-out thresholds of less than 2 ng/L and less than 5 ng/L were 99.5% (95% confidence interval 98.9-100.0%) and 99.6% (99.3-99.8%), respectively.

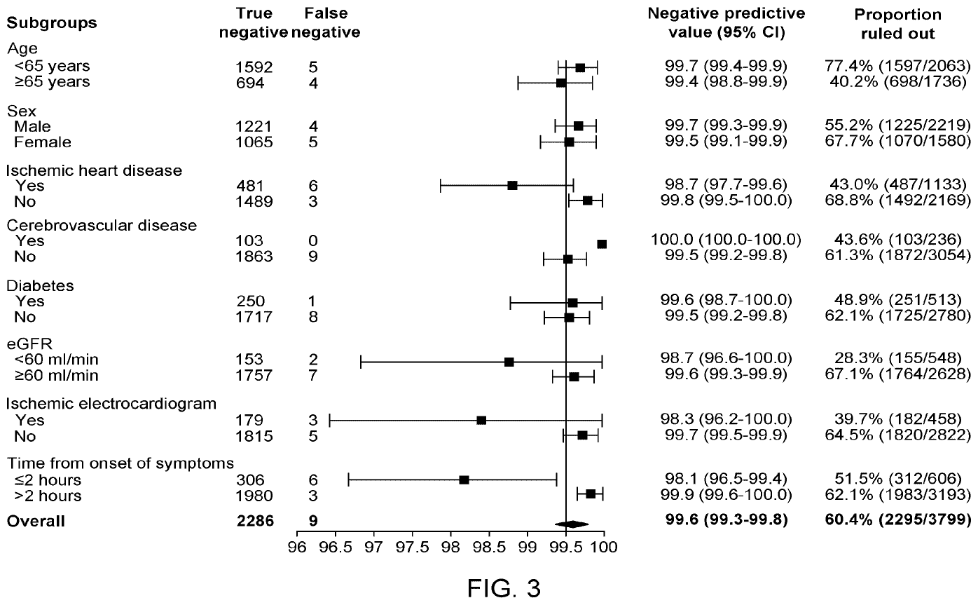

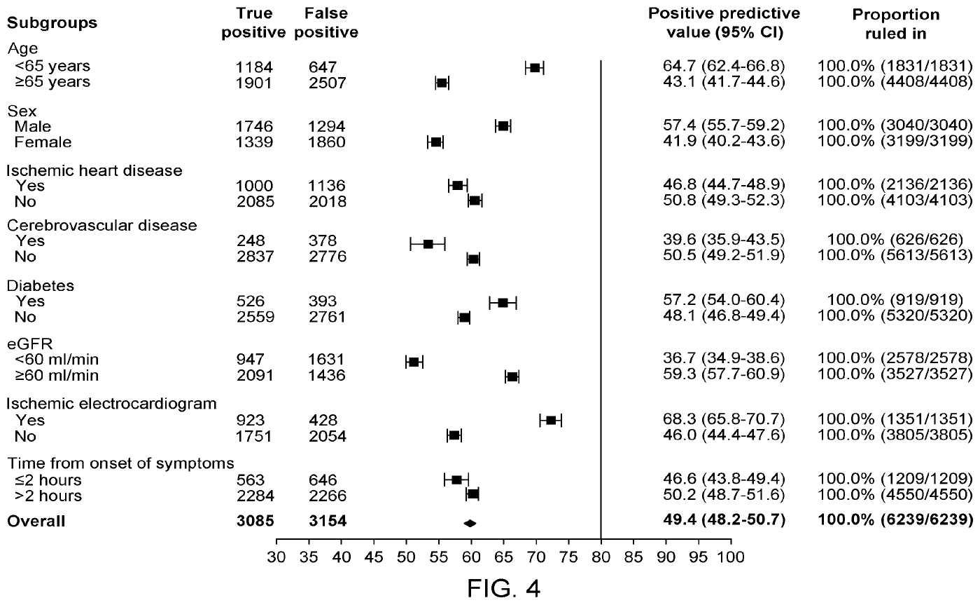

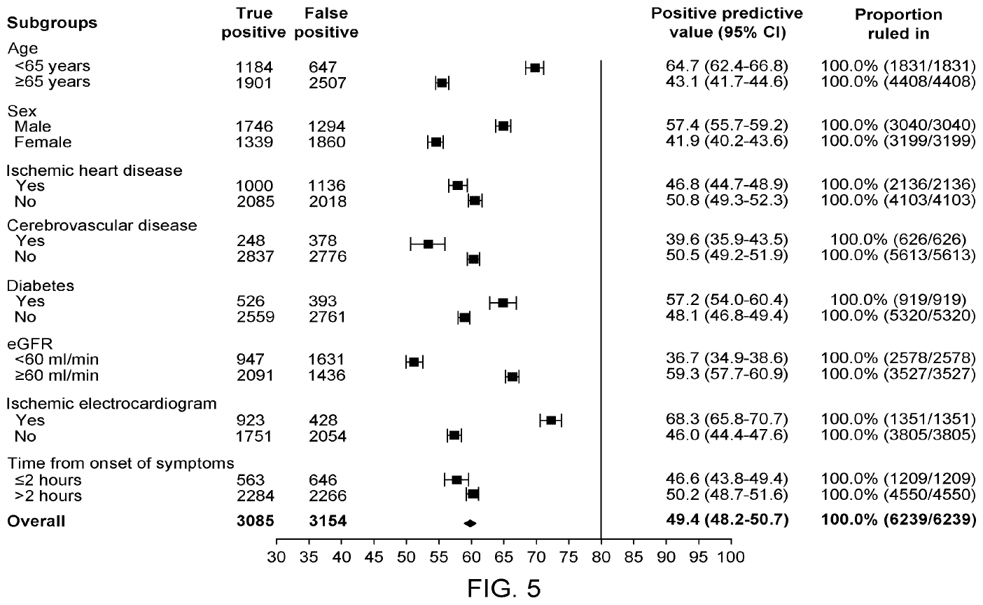

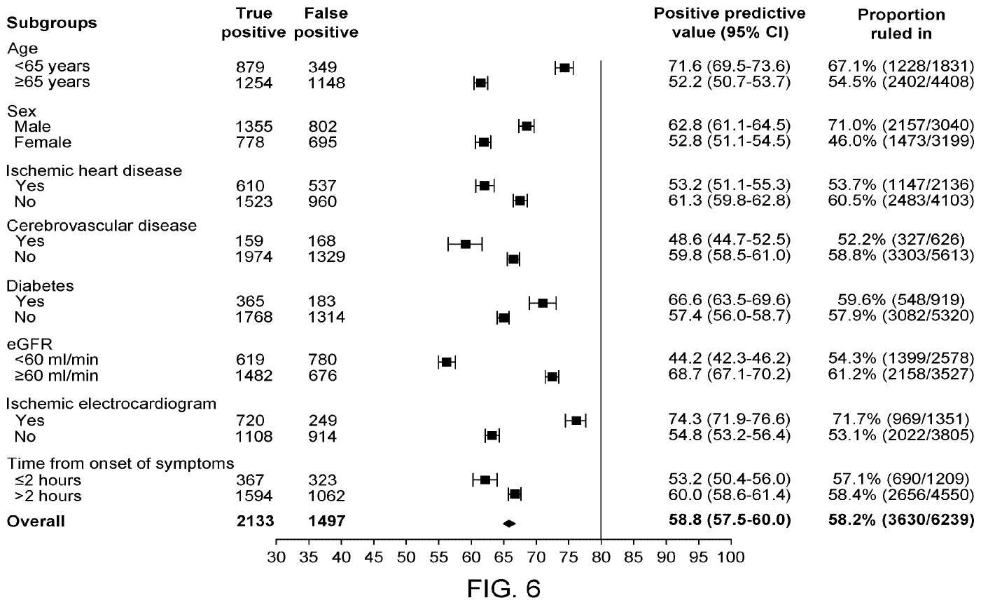

The corresponding sensitivities were 97.7% (94.7-100.0%) and 93.2% (88.8-97.2%). Overall, 16.0% (609/3,799) and 60.4% (2,295/3,799) of subjects without myocardial injury at presentation had troponin concentrations below 2 ng/L and 5 ng/L, respectively. Negative predictive values for both thresholds were lower in subjects presenting within 2 hours of symptom onset and in those with evidence of myocardial ischaemia on the electrocardiogram FIGS. 3 and 4. Among subjects with myocardial injury, the positive predictive value of the sex-specific 99^{th} centile and rule-in threshold of 64 ng/L was 49.4% (48.2-50.7%) and 58.8% (57.2-60.3%), respectively. For both thresholds, there was significant heterogeneity in all subgroups with a lower positive predictive value in those greater than 65 years old, in women, and in those with known ischemic heart disease and impaired renal function, see for example FIGS. 5 and 6.

The XGBoost model is the best performing model in the derivation cohort and therefore this model was selected for the algorithm (hereinafter referred to a CoDE-ACS)

### Table 4. Diagnostic Performance of Statistical Models in the Derivation Cohort

### 8. Rule-Out Diagnostic Performance in Subjects Without Myocardial Injury

| | NPV | Sensitivity | Proportion | | Brier |
|---|---|---|---|---|---|
| | (95% CI) | (95% CI) | ruled out | AUC | score |
| Presentation | | | | | |
| Logistic | 99.8 | 97.7 | 43.7% | 0.898 | 0.028 |
| regression | (99.6-100.0) | (95.0-100.0) | | (0.874-0.923) | |
| Naïve | 99.9 | 99.2 | 38.0% | 0.875 | 0.054 |
| Bayes | (99.7-100.0) | (97.4-100.0) | | (0.850-0.900) | |
| Random | 99.8 | 97.0 | 60.4% | 0.904 | 0.028 |
| Forest | (99.6-100.0) | (93.8-99.3) | | (0.881-0.927) | |
| XGBoost | 99.8 | 96.2 | 66.2% | 0.914 | 0.028 |
| | (99.6-100.0) | (92.3-99.2) | | (0.894-0.934) | |
| Serial | | | | | |
| Logistic | 100.0 | 100.0 | 12.0% | 0.890 | 0.052 |
| regression | (100.0-100.0) | (100.0-100.0) | | (0.860-0.921) | |
| Naïve | 98.0 | 83.5 | 68.8% | 0.870 | 0.062 |
| Bayes | (97.1-98.8) | (76.6-89.4) | | (0.834-0.906) | |
| Random | 99.8 | 98.5 | 54.3% | 0.964 | 0.047 |
| Forest | (99.4-100.0) | (96.0-100.0) | | (0.947-0.981) | |
| XGBoost | 99.8 | 98.5 | 70.4% | 0.973 | 0.031 |
| | (99.5-100.0) | (96.1-100.0) | | (0.961-0.985) | |

### B. Rule-In Diagnostic Performance in Subjects With Myocardial Injury

| | PPV | Specificity | Proportion | | Brier |
|---|---|---|---|---|---|
| | (95% CI) | (95% CI) | ruled in | AUC | score |
| Presentation | | | | | |
| Logistic | 80.0 | 84.2 | 40.0% | 0.845 | 0.160 |
| regression | (78.3-81.6) | (82.9-85.5) | | (0.836-0.855) | |
| Naïve | 80.1 | 84.9 | 38.3% | 0.831 | 0.174 |
| Bayes | (78.4-81.7) | (83.7-86.2) | | (0.821-0.841) | |
| Random | 80.1 | 81.8 | 46.4% | 0.869 | 0.148 |
| Forest | (78.6-81.6) | (80.3-83.2) | | (0.861-0.878) | |
| XGBoost | 80.0 | 83.1 | 42.9% | 0.852 | 0.157 |
| | (78.4-81.5) | (81.7-84.4) | | (0.842-0.861) | |
| Serial | | | | | |
| Logistic | 82.8 | 80.1 | 49.2% | 0.836 | 0.162 |
| regression | (81.2-84.2) | (78.4-81.7) | | (0.825-0.847) | |
| Naïve | 82.2 | 80.1 | 47.5% | 0.824 | 0.194 |
| Bayes | (80.7-83.7) | (78.4-81.7) | | (0.813-0.836) | |
| Random | 83.4 | 80.2 | 50.6% | 0.855 | 0.163 |
| Forest | (82.0-84.8) | (78.5-81.8) | | (0.845-0.865) | |
| XGBoost | 83.2 | 80.1 | 50.3% | 0.850 | 0.155 |
| | (81.7-84.4) | (78.4-81.6) | | (0.840-0.860) | |

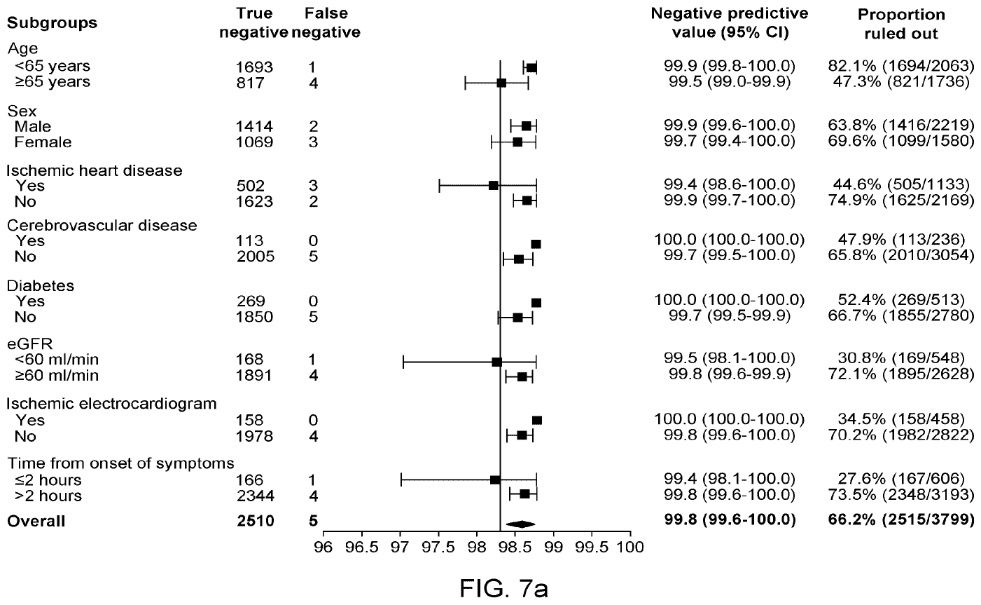

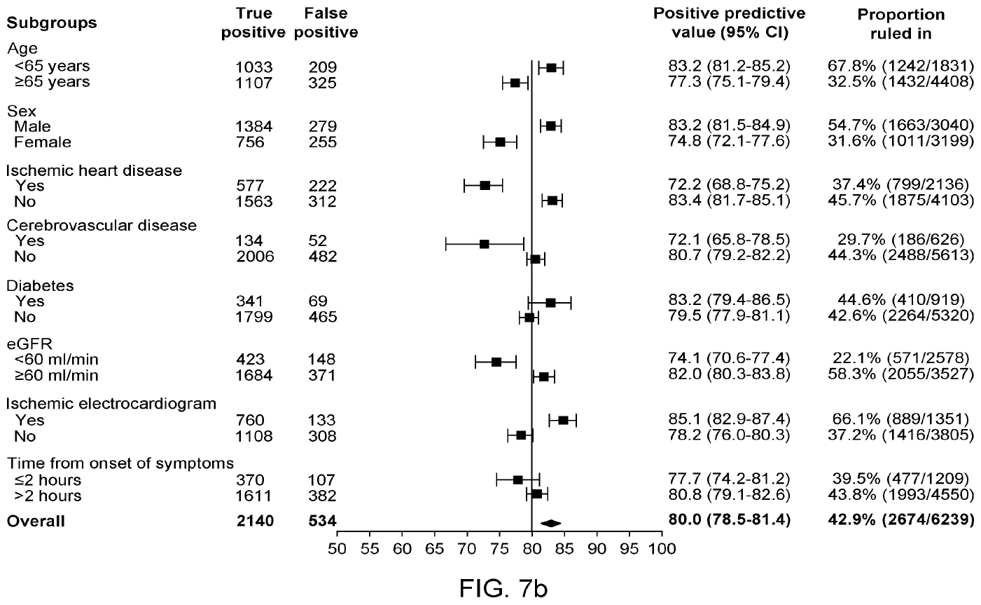

Using high-sensitivity cardiac troponin concentrations at presentation, a CoDE-ACS rule-out score of 1.2 achieved our prespecified diagnostic performance metrics with a negative predictive value of 99.8% (99.6-100.0%) and sensitivity of 96.2% (92.3-99.2%) in those without myocardial injury at presentation. A CoDE-ACS rule-in score of 59.9 achieved a positive predictive value of 80.0% (78.4-81.5) and specificity of 83.1% (81.7-84.4%) in those with evidence of myocardial injury at presentation. Rule-out and rule-in scores had a consistent performance across all important subject subgroups, see for example FIGS. 7a and 7b. If these scores are used to triage subjects with suspected acute coronary syndrome, 66.2% (2,515/3,799) of those without myocardial injury and 42.9% (2,674/6,239) of those with myocardial injury will have myocardial infarction ruled-out and ruled-in respectively at presentation.

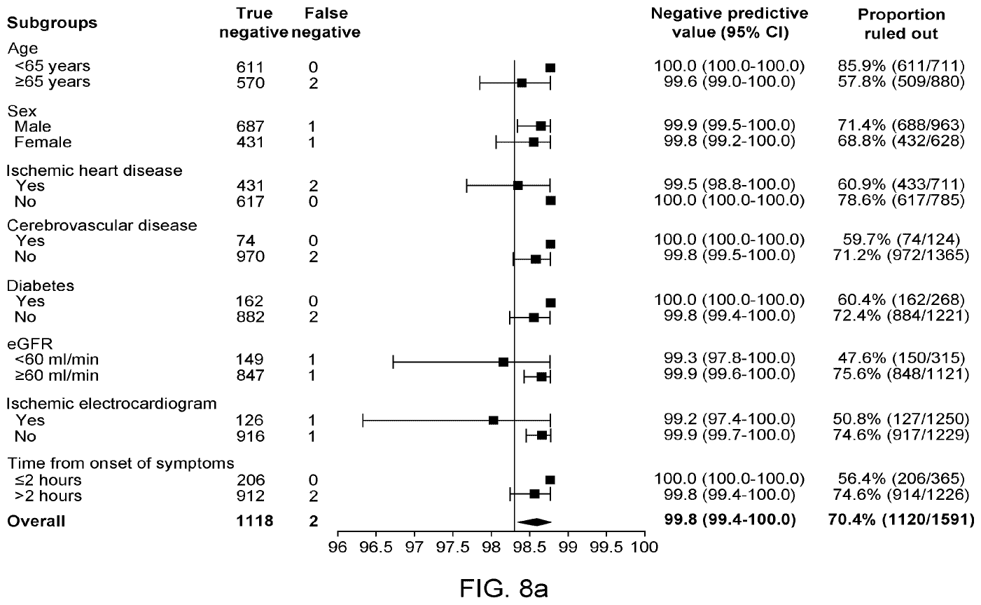

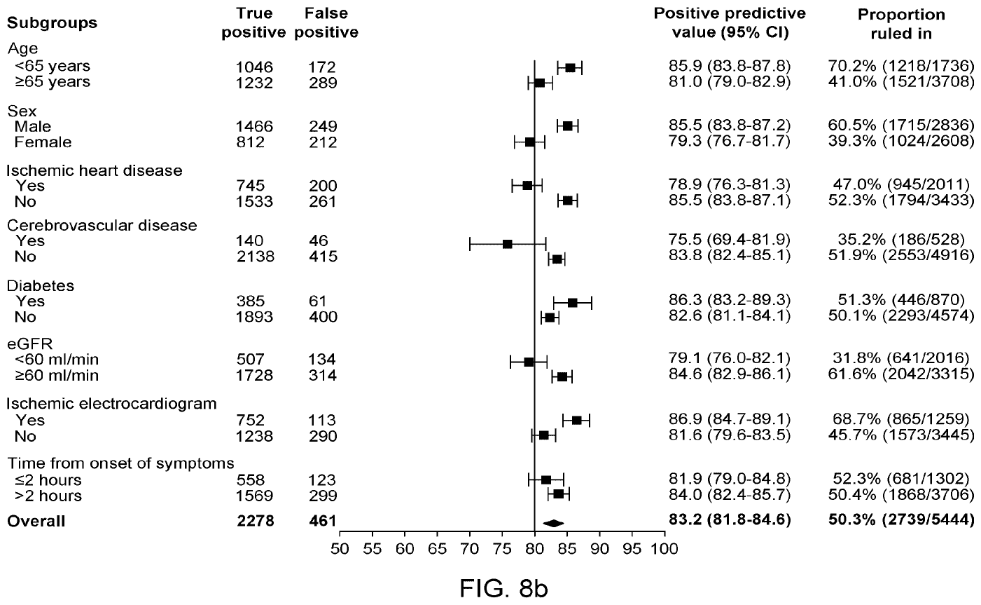

When the first and second cardiac troponin concentrations were incorporated within the CoDE-ACS model, a CoDE-ACS rule-out score of 0.6 achieved a negative predictive value of 99.8% (99.5-100.0) and a sensitivity of 98.5% (96.1-100.0), see for example FIG. 8a. The rule-in performance for the of CoDE-ACS, rule-in score of 62.9, improved with serial cardiac troponin concentrations with a positive predictive of 83.2% (81.7-84.4) and a specificity of 80.1 (78.4-81.6). The positive predictive value of CoDE-ACS was improved across all subject subgroups, see for example FIG. 8b.

### External Validation of CODE-ACS

The pooled external validation cohort consists of 3,035 subjects (median age 57 years, 61% male), see Table 1. During the index hospitalisation, myocardial infarction (type 1, 4b or 4c) occurred in 49% (3,062/6,239) and 3% (132/3,799) in subjects with and without evidence of myocardial injury at presentation.

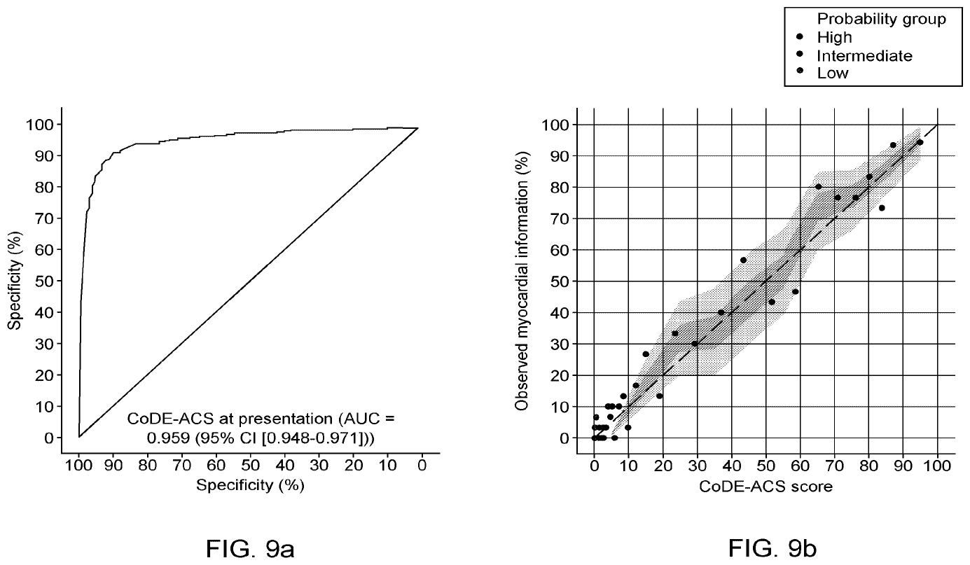

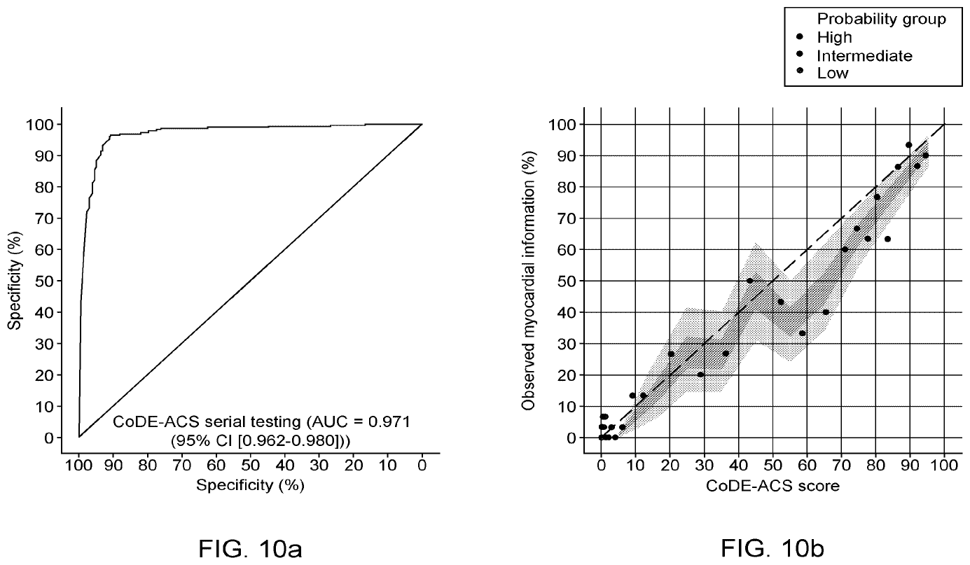

In the pooled external validation cohort, the area under curve for CoDE-ACS was 0.959 (0.948-0.971) using the presentation troponin concentration alone and 0.971 (0.962-0.980) using serial troponin results with good calibration (Brier score of 0.040 and 0.039, respectively, see for example FIGS. 9a and 9b and 10a and 10b. In FIGS. 9b and 10b the dashed line represents perfect calibration, and each point represents 100 subjects. A CoDE-ACS score of 1.2 achieved an NPV of 99.5% (99.1-99.7%) and sensitivity of 84.1% (82.7-85.5%) whilst a score of 59.9 achieved a PPV of 85.1% (81.1-88.3%) and a specificity of 65.7% (60.8-70.3%) with the presentation troponin result.

| | True | False | True | False | NPV | Sensitivity | Proportion |
|---|---|---|---|---|---|---|---|
| | negative | negative | positive | positive | (95% CI) | (95% CI) | ruled out |
| Rule-out CoDE- | | | | | | | |
| ACS threshold | | | | | | | |
| Presentation | 1875 | 10 | 53 | 718 | 99.5 | 84.1 | 71.0% |
| (1.2) | | | | | (99.1-99.7) | (82.7-85.5) | |
| Serial testing | 2030 | 5 | 28 | 527 | 99.8 | 84.8 | 78.6% |
| (0.6) | | | | | (99.5-99.9) | (83.4-86.2) | |

### Table 5. Diagnostic Performance of CODE-ACS in the External Validation Cohort

| | True | False | True | False | PPV | Specificity | Proportion |
|---|---|---|---|---|---|---|---|
| | negative | negative | positive | positive | (95% CI) | (95% CI) | ruled in |
| Rule-in CoDE- | | | | | | | |
| ACS threshold | | | | | | | |
| Presentation | 71 | 60 | 211 | 37 | 85.1 | 65.7 | 65.4% |
| (59.9) | | | | | (81.1-88.3) | (60.8-70.3) | |
| Serial testing | 74 | 52 | 249 | 70 | 78.1 | 51.4 | 71.7% |
| (62.9) | | | | | (74.0-81.7) | (46.8-56.0) | |

### A. Rule-Out Thresholds in Subjects Without Myocardial Injury

### B. Rule-In Thresholds in Subjects With Myocardial Injury

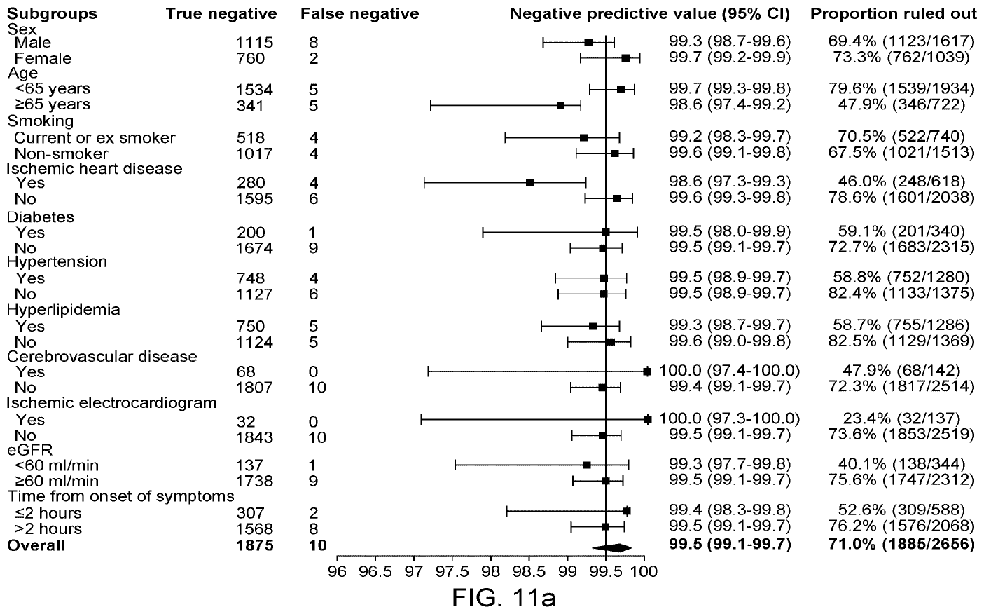

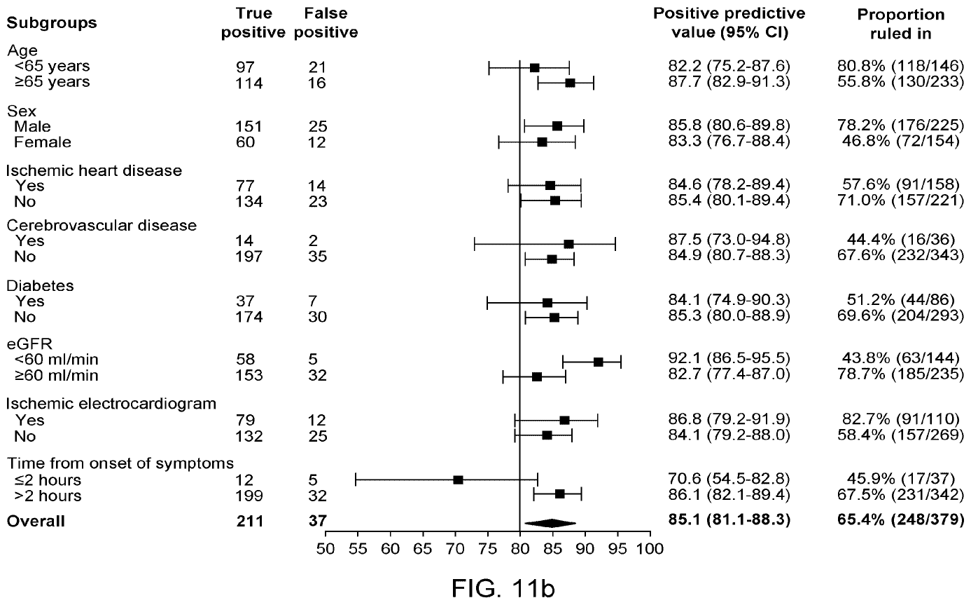

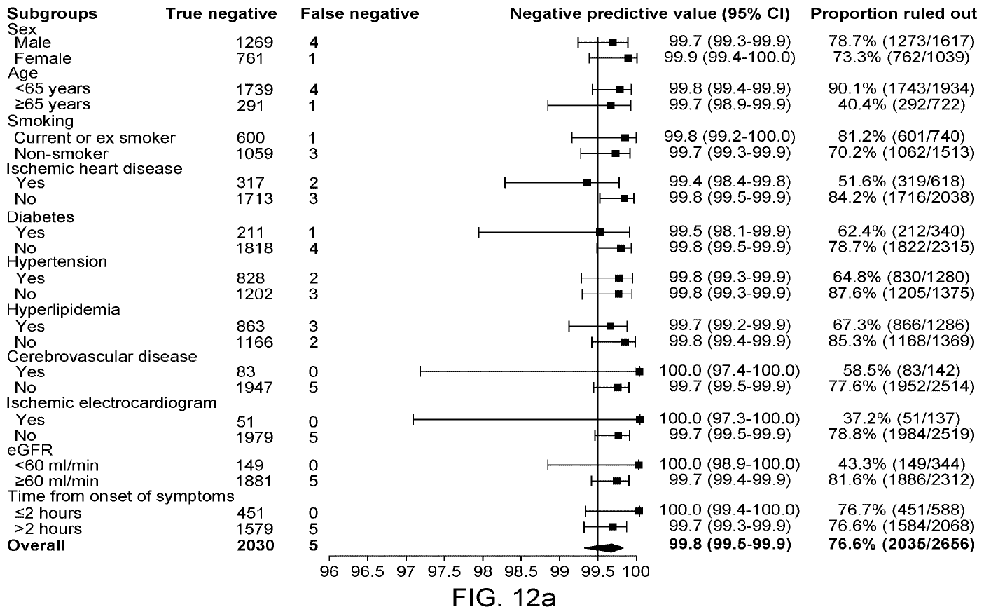

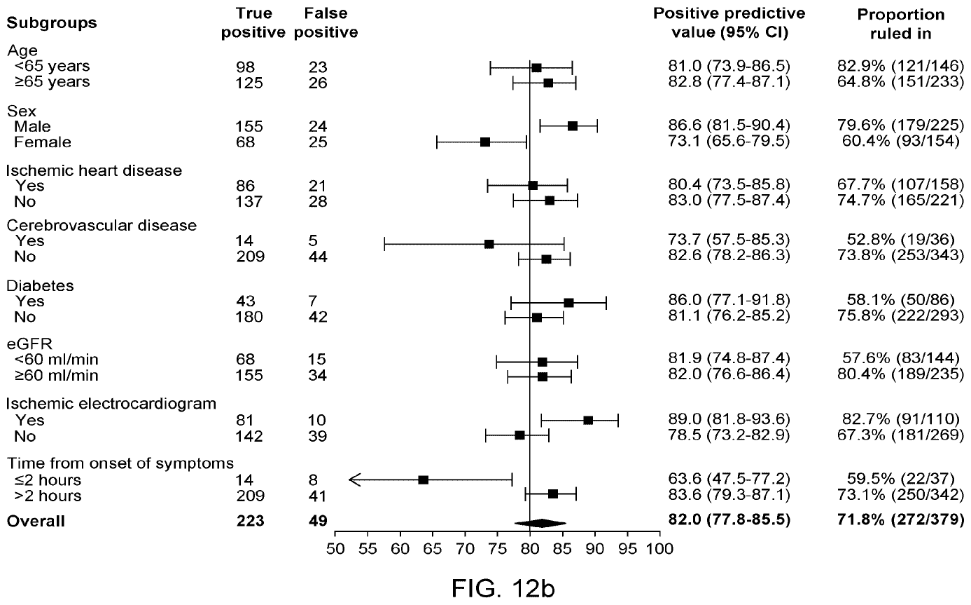

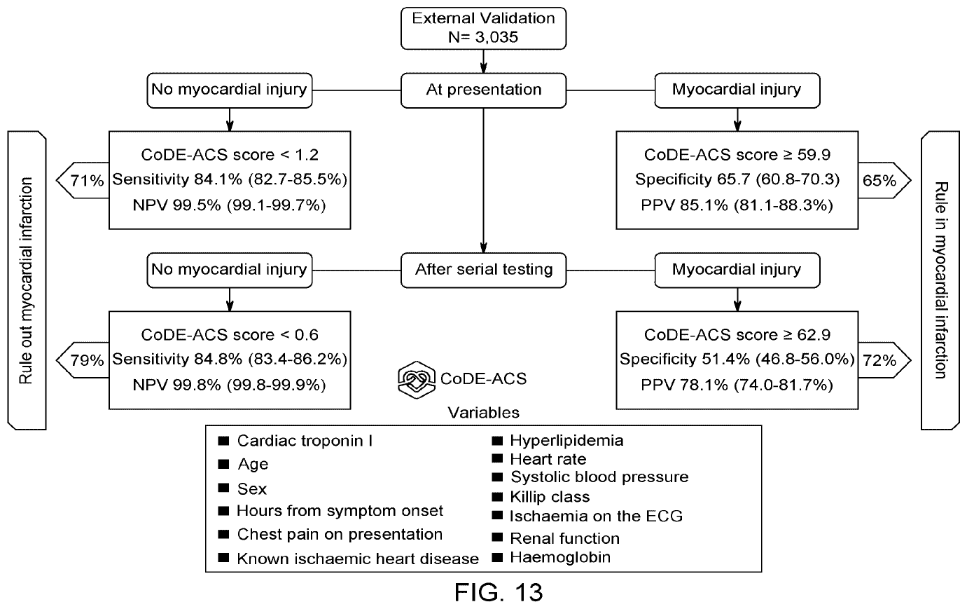

These rule-in and rule-out scores had similar diagnostic performance across all subgroups, see for example FIGS. 11a and 11b. If these scores were applied in subjects with suspected acute coronary syndrome, CoDE-ACS would identify 71% (1,885/2,656) of subjects without myocardial injury at presentation at low-probability (<1.2) and 65.4% (248/379) of subjects with myocardial injury at presentation at high-probability (>59.9) of myocardial infarction. With serial troponin concentrations, CoDE-ACS had improved performance to rule-out (NPV of 99.8% [99.5-99.9%] and sensitivity of 84.8% [83.4-86.2%]) and rule-in (PPV of 78.1% [74.0-81.7%] and specificity of 51.4% [46.8-56.0%]) myocardial infarction, see for example FIGS. 12a, 12b and 13.

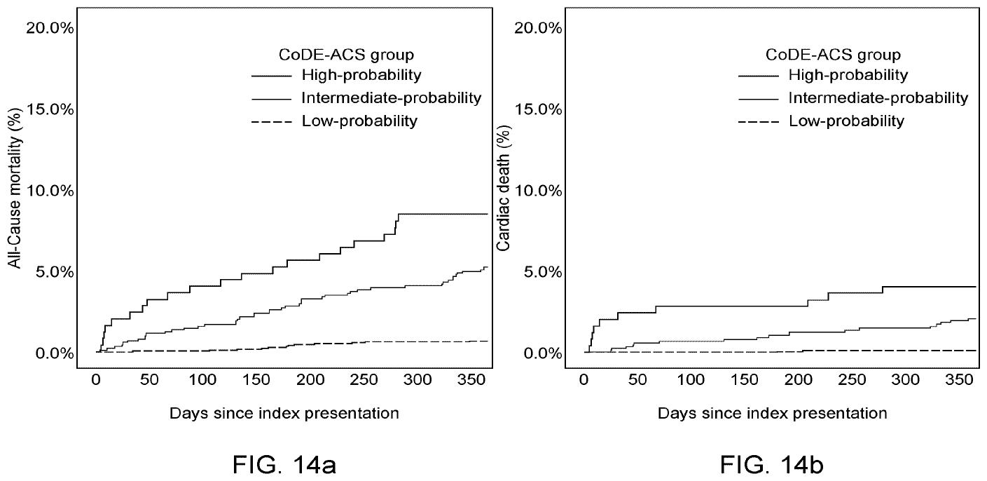

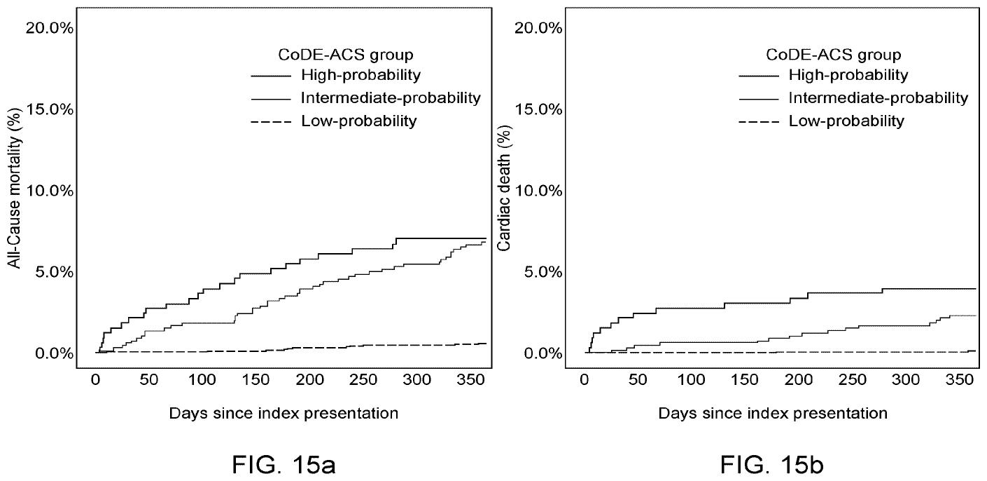

Subjects who were identified as low-probability for myocardial infarction at presentation had a lower rate of all-cause and cardiac mortality compared to those with an intermediate- and high-probability at 30-days (all-cause mortality: 0.0% versus 0.7% and 2.0% respectively; cardiac mortality: 0% versus 0.1% and 1.8%) and 1 year (all-cause mortality: 0.6% versus 5.1% and 8.3% respectively; cardiac mortality: 0.1% versus 2.2% and 3.9%, respectively), see for example FIGS. 14a and b. Similar associations are observed in the model incorporating serial troponin measurements, see for example FIGS. 15 a and b.

The use of statistical modelling has several important advantages over the use of fixed troponin thresholds alone. Cardiac troponin is known to be influenced by various factors such as sex, age, renal function and comorbidity burden which explains the heterogeneity in diagnostic performance in subgroups observed in this study. Furthermore, subject with different patterns of comorbidity have different pre-test probability of having myocardial infarction. The CoDE-ACS provided consistent diagnostic performance across these subject subgroups by incorporating subject factors using machine learning. In contrast to current clinical pathways, where subjects presenting within 3 hours of symptom onset or with myocardial ischemia on the electrocardiogram are excluded, CoDE-ACS can be applied in all subject presenting to the Emergency Department irrespective of their symptom onset or risk profile. Furthermore, machine learning enables serial troponin measurements to be performed at a second flexible timepoint. At present, all guideline recommended pathways require serial cardiac troponin measurement at fixed timepoints, which is challenging to implement precisely in clinical practice. Whilst previous studies have showed that adherence to these pathways was high, one in five to one in three subjects did not undergo troponin testing in accordance with the pathway recommendations which may significantly impact on the accuracy and safety of assessment. Finally, pathways that rely on fixed thresholds may perform variably across healthcare systems due to differences in the way cardiac troponin testing is performed and differences in the prevalence of myocardial infarction. The advantage of using a decision-support tool, such as CoDE-ACS, that generates probabilities of myocardial infarction for individual subjects and estimates the diagnostic performance associated with these probabilities is that healthcare systems can apply the algorithm more flexibly. For example, in a healthcare setting that is more conservative, lower CoDE-ACS values could be used to maximise the negative predictive value or in those healthcare settings with less capacity for assessment in the Emergency Department higher CoDE-ACS values could be applied that reduce the number of subjects who are neither ruled out or ruled in but triaged to a period of observation.

It will be appreciated that, CoDE-ACS was developed and validated using a high-sensitivity cardiac troponin I assay. As all cardiac troponin assays are not standardised, CoDE-ACS will need to be trained and validated across each different assay separately. Even allowing for significant differences in the demographic and subject characteristics between our derivation and external validation cohorts, the CoDE-ACS had excellent performance in the external validation cohorts despite these significant differences.

1. A computer implemented method of identifying an subject's likelihood of having myocardial infarction comprising the steps of operating upon data corresponding to the level of a cardiac biomarker using point of care and/or core laboratory assays in at least one sample from an individual with at least two other data elements indicative of respective clinical indicators from the individual in a statistical model to compute the probability of myocardial infarction for the individual wherein the data corresponding to the level of the cardiac biomarker is provided as a discrete variable in the model.

2. The computer implemented method of claim 1, wherein the data corresponding to the level of the cardiac biomarker in at least one sample from an individual comprises data corresponding to the level of the cardiac biomarker in a single sample.

3. The computer implemented method of claim 2, wherein the clinical parameters may comprise at least two data elements are selected from the list comprising: age, sex, the number of hours from symptom onset to cardiac troponin measurement, presenting symptoms, prior medical diagnoses, such as known ischaemic heart disease, hyperlipidemia and other risk factors, heart rate, blood pressure, Killip class, information from an electrocardiogram, renal function, haemoglobin and other information from laboratory testing or imaging. Renal function may be estimated by glomerular filtration rate calculated using the Chronic Kidney Disease Epidemiology Collaboration formula.

4. The computer implemented method of claim 1. further comprising loading respective training data sets corresponding to subjects with and without myocardial injury into a machine learning system wherein the machine-learning system includes a processing circuitry arranged to be trained to myocardial infarction using the training data within the statistical model.

5. The computer implemented method of claim 1, further comprising training the machine learning system by performing a plurality of iterations of 10-fold cross-validation respective training data sets corresponding to subjects with and without myocardial injury to compute a score to indicative of the probability of having myocardial infarction for each individual in the respective training data sets.

6. The computer implemented method of claim 4, further comprising using the machine learning system to execute the statistical model on the data corresponding to the level of the cardiac biomarker in at least one sample from an individual with at least two other data elements indicative of respective clinical indicators from the individual once trained.

7. The computer implemented method of claim 1, wherein the statistical model may comprise an XGBoost model from the boosting family of models or a random forest model from the bagging family of models or artificial and/or convolutional neural networks models or logistic regression or generalised linear mixed models. For the XGBoost model, a probability is computed by performing an inverse-logit transformation of the sum of the weights of the terminal nodes of the trained model
$${\hat{y}}_{i} = {\sum }_{k = 1}^{K} {f}_{k} ( {x}_{i} ) , {f}_{k} ∈ F$$
where f is an function that map each variable vector xᵢ (xᵢ={xᵢ, x₂, . . . , xₙ}, i=1, 2, N) to the outcome yᵢ, K is the number of Classification and Regression Trees (CART) and F is the space of function containing all CART.

8. The method of claim 1, further comprising generating a probability score for an individual that would classify the individual as a high-, intermediate- or a low-probability of myocardial infarction.

9. The method of claim 1, further comprising defining one or more user variable predictor values based upon a user input.

10. The method of claim 9, wherein the one or more user variable predictor variables define respective thresholds for classifying an individual as a high- or a low-probability of myocardial infarction.

11. The method of claim 1, wherein the cardiac biomarker comprises cardiac troponin I and/or cardiac troponin T, natriuretic peptides and/or cardiac myosin binding protein C measured using point of care and/or core laboratory assays.

12. (canceled)

13. A system for identifying a subject's likelihood of having myocardial infarction comprising:
a processor;
a data storage device; and
an output device;
wherein the processor is arranged to receive data corresponding to the level of a cardiac biomarker in at least one sample from an individual with at least two other data elements indicative of respective clinical indicators from the individual in a statistical model from the data storage device and to execute a set of instructions to cause the processor to:
operate upon data corresponding to the level of the cardiac biomarker in the at least one sample from an individual with the at least two other data elements indicative of respective clinical indicators from the individual in a statistical model to compute the probability of myocardial infarction for the individual wherein the data corresponding to the level of the cardiac biomarker is provided as a discrete variable in the model; and
output data corresponding to the likelihood of the individual having myocardial infarction at the output device.

14. The system of claim 13, wherein the data corresponding to the level of the cardiac biomarker in at least one sample from an individual comprises data corresponding to the level of the cardiac biomarker in a single sample.

15. The system of claim 13, wherein the clinical parameters comprise at least two data elements are selected from the list comprising: age, sex, the number of hours from symptom onset to cardiac troponin measurement, presenting symptoms, prior medical diagnoses, such as known ischaemic heart disease, hyperlipidemia and other risk factors, heart rate, blood pressure, Killip class, information from an electrocardiogram, renal function, haemoglobin and other information from laboratory testing or imaging. Renal function may be estimated by glomerular filtration rate calculated using the Chronic Kidney Disease Epidemiology Collaboration formula.

16. The system of claim 13, wherein the processor is arranged to load respective training data sets corresponding to subjects with and without myocardial injury from the data storage device into a machine learning sub-system system wherein the machine-learning sub-system system includes a processing circuitry arranged to be trained to myocardial infarction using the training data within the statistical model.

17. The system of claim 16, wherein the machine learning system sub-system is arranged to be trained by performing a plurality of iterations of 10-fold cross-validation respective training data sets corresponding to subjects with and without myocardial injury to compute a score to indicative of the probability of having myocardial infarction for each individual in the respective training data sets.

18. The system of claim 16, wherein the machine learning sub-system is arranged to execute instructions that cause the statistical model to be executed on the data corresponding to the level of the cardiac biomarker in at least one sample from an individual with at least two other data elements indicative of respective clinical indicators from the individual once the machine learning sub-system is trained.

19. The system of claim 13, wherein the statistical model comprises an XGBoost model wherein a probability that is computed by performing an inverse-logit transformation of the sum of the weights of the terminal nodes of the trained model, the XGBoost model
$${\hat{y}}_{i} = {\sum }_{k = I}^{K} {f}_{k} ( {x}_{i} ) , {f}_{k} ∈ F$$
where f is an function that map each variable vector xᵢ (xᵢ={xᵢ, x₂, . . . , xₙ}, i=1, 2, N) to the outcome yᵢ, K is the number of Classification and Regression Trees (CART) and F is the space of function containing all CART.

20. The system of claims 13, wherein the processor is arranged to generate a probability score for an individual that would classify the individual as a high-, intermediate- or a low-probability of myocardial infarction.

21. The system of claim 13, wherein, the processor is arranged to define one or more user variable predictor values based upon a user input.

22. The system of claim 21, wherein the one or more user variable predictor variables may define thresholds for classifying an individual as a high-, intermediate- or low-probability of myocardial infarction.

23. The system of claim 13, wherein the cardiac biomarker comprises cardiac troponin I and/or cardiac troponin T, natriuretic peptides and/or cardiac myosin binding protein C measured using point of care and/or core laboratory assays.

24. The system of claim 13, wherein the data corresponding to the level of a cardiac biomarker is acquired using a point of care and/or a core laboratory assay.

25. A processor arranged to execute the method of identifying an subject's likelihood of having myocardial infarction comprising the steps of operating upon data corresponding to the level of a cardiac biomarker using point of care and/or core laboratory assays in at least one sample from an individual with at least two other data elements indicative of respective clinical indicators from the individual in a statistical model to compute the probability of myocardial infarction for the individual wherein the data corresponding to the level of the cardiac biomarker is provided as a discrete variable in the model.

26. A computer implemented tool capable of receiving data to allow establishment or the ruling out of a risk of myocardial infarction comprising the processor of claim 25.
